(19) United States
(12) Patent Application Publication
(10) Pub. No.: US 2010/0159508 A1
(43) Pub. Date: Jun. 24, 2010

(54) THERMALLY RESPONSIVE MICELLES

(75) Inventors: Yi-Yan Yang, Nanos (SG); Wei Liu Hong, Nanos (SG); Chi-Bun Ching, Nanos (SG); Chen Hong, Nanos (SG)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: AGENCY FOR SCIENCE, TECHNOLOGY AND RESEARCH, Singapore (SG); NANYANG TECHNOLOGICAL UNIVERSITY, Singapore (SG)

(21) Appl. No.: 12/307,357

(22) PCT Filed: Jul. 6, 2007
(86) PCT No.: PCT/SG2007/000199

§ 371 Date: Mar. 9, 2010

Related U.S. Application Data
(60) Provisional application No. 60/818,522, filed on Jul. 6, 2006.

(51) Int. Cl. C12N 9/96; C08F 20/56; C08F 226/02; B01J 13/02; C12P 1/00

(52) U.S. Cl. 435/41; 435/188; 526/303.1; 526/307.6; 526/307.7; 264/4.1

(57) ABSTRACT

The invention provides an amphiphilic copolymer comprising monomer units derived from a first monomer and monomer units derived from a second monomer. The copolymer has at least one hydrophobic endgroup. The first monomer is such that the copolymer is thermally responsive and the second monomer comprises a carboxylic acid or carboxylate group.

### TECHNICAL FIELD

The present invention relates to thermally responsive micelles and to processes for making them.

### BACKGROUND OF THE INVENTION

Enzymes have a variety of biological, biomedical and pharmaceutical applications. In particular, they are being increasingly exploited as biocatalysts for the synthesis of pharmaceuticals and fine chemicals because they provide high enantio- and regio-selectivity, and are more environmentally friendly. However, the use of enzymes is limited due to their unstable nature and the stringent requirements for their surrounding environment. Extremely low or high pH, high temperature and the presence of organic solvents may lead to the denaturation of enzymes.

Therefore, many approaches have been proposed to improve enzyme stability, including enzyme immobilization or encapsulation, enzyme modification and medium engineering. Among these approaches, enzyme immobilization or encapsulation is the most commonly explored and efficient method because of the possibility of recycling and continuous operation, and the ease in product purification. Enzymes have been immobilized into mesoporous matrices such as silica and polysaccharide, attached to nanoparticles and polymer nanofibers.

Reversed micelles have also been widely studied for enzyme encapsulation as they enable enzymatic reactions in organic solvents, which is important in the synthesis of many chiral pharmaceuticals. Reversed micelles in general possess a core-shell structure. The hydrophilic core is used for the immobilization/encapsulation of enzymes, providing a favorable aqueous environment for achieving high enzyme activity. The hydrophobic shell makes the micelles soluble or dispersible in organic solvents, and prevents direct contact of the enclosed enzymes with unfavourable organic solvents. This therefore enhances the stability of the encapsulated enzymes. In addition, the micelles of around micron size provide large interfacial area, reducing or eliminating mass-transfer barriers of substrates and thus enhancing the enzyme activity.

Reversed micelles reported in the literature have been fabricated from conventional ionic and nonionic surfactants including sodium bis(2-ethylhexyl) sulfosuccinate (AOT), cetyltrimethylammoniumbromide (CTAB) and polyoxyethylene sorbitan trioleate (Tween 85). Strong electrostatic and hydrophobic interactions between the ionic reversed micelles and the enzymes reduced the activity and stability of the enzymes. Therefore, nonionic surfactants such as Tween 85 have been added as a co-surfactant to decrease the interface charge density and the hydrophobicity of the ionic reversed micelles. Although micelles formed from nonionic surfactants were also found to provide the high activity and stability of enzymes, co-surfactants were necessary for the formation of the micelles. AOT has been modified by inserting a hydrophilic polyoxyethylene group between the head group and the hydrophobic tail of AOT. This modified AOT significantly increased the activity and stability of the enzyme lipase.

Although reversed micelles provide many advantages over other enzyme immobilization or encapsulation systems, conventional reversed micelles present a major disadvantage associated with the presence of high concentrations of low molecular mass surfactants, which causes difficulties in product separation and enzyme recovery.

### OBJECT OF THE INVENTION

It is the object of the present invention to substantially overcome or at least ameliorate one or more of the above disadvantages.

### SUMMARY OF THE INVENTION

In a broad form of the invention there is provided an amphiphilic copolymer comprising:
monomer units derived from a first monomer;
monomer units derived from a second monomer, said second monomer being ionic or ionisable; and
at least one hydrophobic endgroup;
   wherein the first monomer is such that the copolymer is capable of forming micelles in a hydrophobic liquid. The micelles may be capable of encapsulating a biological substance.

The first monomer may be such that the copolymer is thermally responsive. The second monomer may be anionic or may be acidic.

The invention also provides processes for making the copolymer by endcapping a precursor copolymer comprising the first and second monomer units with an endcapping reagent comprising the hydrophobic endgroup. It also provides micellar solutions comprising micelles of the amphiphilic copolymer, and processes for making them by micellisation of the amphiphilic copolymer in an organic liquid. The micellar solutions may also comprise a biological substance, for example an enzyme, located in the micelles. The invention also provides a method for conducting a reaction comprising exposing reagents to the micelles, wherein a biological species for catalysing the reaction is located in the micelles.

In a first aspect of the invention there is provided an amphiphilic copolymer comprising:
monomer units derived from a first monomer;
monomer units derived from a second monomer, said second monomer comprising a carboxylic acid or carboxylate group; and
at least one hydrophobic endgroup;
   wherein the first monomer is such that the copolymer is thermally responsive.

The following options may be used with the first aspect, or with the broad form of the invention stated above, either individually or in any suitable combination.

The first monomer may be an N-alkylacrylamide. It may be N-isopropylacrylamide.

The second monomer may be selected from the group consisting of acrylic acid, methacrylic acid, acrylate and methacrylate.

The hydrophobic endgroup may be a C1 to C24 straight chain alkyl group or a C3 to C24 branched chain alkyl group. It may be an octadecyl group.

The hydrophobic endgroup may be coupled to one of the monomer units by a —S(CH₂)ₙO— group. n may be between 2 and about 24.

In an embodiment there is provided an amphiphilic copolymer comprising:
monomer units derived from N-isopropylacrylamide;
monomer units derived from a second monomer, said second monomer comprising a carboxylic acid or carboxylate group; and
at least one hydrophobic endgroup.

In another embodiment there is provided an amphiphilic copolymer comprising:
monomer units derived from N-isopropylacrylamide;
monomer units derived from acrylic acid or acrylate; and
at least one hydrophobic endgroup.

In another embodiment there is provided an amphiphilic copolymer comprising:
monomer units derived from N-isopropylacrylamide;
monomer units derived from acrylic acid or acrylate; and
a C12 to C18 alkyl endgroup.

In another embodiment there is provided an amphiphilic copolymer comprising:
monomer units derived from N-isopropylacrylamide;
monomer units derived from acrylic acid or acrylate; and
a C12 to C18 alkyl endgroup, said endgroup coupled to one of the monomer units by a —S(CH₂)ₙO— group.

In a second aspect of the invention there is provided a micellar solution comprising micelles of a copolymer according to the first aspect in a liquid.

The following options may be used with the second aspect either individually or in any suitable combination.

The micelles may be reverse micelles.

The liquid may be an organic liquid.

The micelles may comprise a core-shell structure in which a hydrophilic core is surrounded by a hydrophobic shell. The hydrophobic endgroups (or at least some thereof) may be located in the shell and the monomer units (or at least some thereof) derived from the second monomer may be located in the core.

There may be a biological substance located in the core of the micelles. The biological substance may be an enzyme. The biological substance may be catalytically active.

In an embodiment there is provided a micellar solution comprising micelles of a copolymer according to the first aspect in an organic liquid whereby the micelles comprise a core-shell structure in which the hydrophobic endgroups are located in the shell and the monomer units derived from the second monomer are located in the core.

In another embodiment there is provided a micellar solution comprising micelles of a copolymer according to the first aspect in an organic liquid whereby the micelles comprise a core-shell structure in which the hydrophobic endgroups are located in the shell and the monomer units derived from the second monomer are located in the core, wherein an enzyme is located in the core of the micelles.

In a third aspect of the invention there is provided a process for making an amphiphilic copolymer comprising the step of
coupling a precursor copolymer to an endcapping reagent so as to attach a hydrophobic endgroup to the precursor polymer to form the amphiphilic copolymer,
   wherein the precursor copolymer is a copolymer of a first monomer and a second monomer and the endcapping reagent comprises the hydrophobic endgroup, said first monomer being such that the amphiphilic copolymer is thermally responsive and said second monomer comprising a carboxylic acid or carboxylate group.

The following options may be used with the third aspect either individually or in any suitable combination.

The process may additionally comprise the step of copolymerising the first monomer and the second monomer by a free radical polymerisation to form the precursor copolymer. The step of copolymerising may be conducted in the presence of a chain transfer agent. The chain transfer agent may comprise a functional group capable of coupling to the endcapping reagent.

The precursor copolymer may have a hydroxyl endgroup. The endcapping reagent may comprise a halogen.

In an embodiment there is provided a process for making an amphiphilic copolymer comprising the steps of:
copolymerising a first monomer and a second monomer by a free radical polymerisation to form a precursor copolymer;
coupling the precursor copolymer to an endcapping reagent so as to attach a is hydrophobic endgroup to the precursor copolymer to form the amphiphilic copolymer,
   wherein the endcapping reagent comprises the hydrophobic endgroup, said first monomer being such that the amphiphilic copolymer is thermally responsive and said second monomer comprising a carboxylic acid or carboxylate group.

In another embodiment there is provided a process for making an amphiphilic copolymer comprising the steps of
copolymerising a first monomer and a second monomer by a free radical polymerisation in the presence of a chain transfer agent, said chain transfer agent comprising a hydroxyl group, to form a precursor copolymer;
coupling the precursor copolymer to an endcapping reagent comprising a halogen so as to attach a hydrophobic endgroup to the precursor copolymer to form the amphiphilic copolymer,
   wherein the endcapping reagent comprises the hydrophobic endgroup, said first monomer being such that the amphiphilic copolymer is thermally responsive and said second monomer comprising a carboxylic acid or carboxylate group.

The invention also provides an amphiphilic copolymer when made by the process of the third aspect.

In a fourth aspect of the invention there is provided a process for making a micellar solution comprising the step of combining an amphiphilic copolymer according to the first aspect and a liquid so as to form micelles of the amphiphilic copolymer in the liquid.

The liquid may be an organic liquid, whereby the micelles adopt a core-shell structure in which a hydrophilic core is surrounded by a hydrophobic shell. The hydrophobic endgroups (or at least some thereof) may be located in the shell and the monomer units (or at least some thereof) derived from the second monomer may be located in the core. The method may comprise allowing the amphiphilic copolymer to self-assemble to form the micelles.

In a fifth aspect of the invention there is provided a process for making a micellar solution comprising the steps of
combining an amphiphilic copolymer according to the first aspect and an organic liquid so as to form micelles of the copolymer in the liquid, whereby the micelles adopt a core-shell structure in which a hydrophilic core is surrounded by a hydrophobic shell; and
adding a biological substance to the organic liquid so as to form the micellar solution wherein the biological substance is located in the core of the micelles.

The biological substance may be added in a second liquid. The biological substance may be dissolved in the second liquid. It may be suspended in the second liquid. It may be emulsified in the second liquid. It may be microemulsified in the second liquid. It may be dispersed in the second liquid. The second liquid may be an aqueous liquid. The hydrophobic endgroups (or at least some thereof) may be located in the shell and the monomer units (or at least some thereof) derived from the second monomer may be located in the core.

In an embodiment there is provided a process for making a micellar solution comprising the steps of:
combining an amphiphilic copolymer according to the first aspect and an organic liquid so as to form micelles of the copolymer in the liquid, whereby the micelles adopt a core-shell structure in which a hydrophilic core is surrounded by a hydrophobic shell; and
adding an enzyme, optionally a solution of an enzyme, in an aqueous liquid to the organic liquid so as to form the micellar solution wherein the enzyme is located in the core of the micelles.

The invention also provides a micellar solution when made by the process of the fourth aspect or the fifth aspect.

In a sixth aspect of the invention there is provided a method for separating a biological substance from a micellar solution, wherein the micellar solution comprises micelles of an amphiphilic copolymer according to the first aspect in an organic liquid, said micelles comprising a core-shell structure in which a hydrophilic core is surrounded by a hydrophobic shell and the biological substance is located in the core of the micelles, said method comprising the step of heating the micellar solution to a temperature above the lower critical solution temperature of the copolymer.

In a seventh aspect of the invention there is provided a method for conducting a reaction of at least one reagent to produce a product, said method comprising the step of combining said at least one reagent with a micellar solution, said micellar solution comprising micelles of an amphiphilic copolymer according to the first aspect in an organic liquid, whereby the micelles comprise a core-shell structure in which a hydrophilic core is surrounded by a hydrophobic shell and a biological substance is located in the core of the micelles, said biological substance being capable of catalysing the reaction.

The method may comprise making the micellar solution according to the process of the fifth aspect of the invention.

The biological substance may be an enzyme, whereby the method is a method for conducting an enzymatic reaction.

The method of the seventh aspect may additionally comprise the step of separating the biological substance from the micellar solution by heating the micellar solution to a temperature above the lower critical solution temperature of the copolymer, said step being conducted after at least some of the at least one reagent has been reacted to produce the product.

The invention also provides a micellar solution, or an amphiphilic copolymer, when used in the method of the seventh aspect.

In an eighth aspect of the invention there is provided a product when produced by the method of the seventh aspect. The product may be an ester. It may be a metabolite.

### DETAILED DESCRIPTION OF THE INVENTION

The invention provides thermally responsive reversed micelles for immobilization/encapsulation of enzymes. The micelles described herein provide improved stability compared to conventional ionic and non-ionic surfactant micelles. The immobilized/encapsulated enzymes may be recovered by simply increasing the environmental temperature. This system has a great potential in immobilizing/encapsulating enzymes for the synthesis of chiral pharmaceuticals.

The present invention provides an amphiphilic copolymer comprising monomer units derived from a first monomer, monomer units derived from a second monomer, and at least one hydrophobic endgroup.

The copolymer is amphiphilic, i.e. it contains at least one hydrophilic region and at least one hydrophobic region. It may be a polymeric surfactant. It may be a random copolymer, or it may be a block copolymer or it may be an alternating copolymer. It may be a combination of these, for example it may have one or more homopolymer blocks and one or more alternating copolymer blocks. The copolymer may have both types of monomer units in the main chain of the polymer. The copolymer may be a linear, or substantially linear, copolymer. The copolymer distribution may depend on the nature of the monomers from which it is made. It should be understood that when reference is made herein to a monomer unit “derived from” a particular monomer, this does not necessarily mean that the particular monomer was the direct precursor of the monomer unit, rather that the monomer unit could have been made from that monomer unit. Thus for example a monomer unit —CH₂—CH(CO₂H)— may be said to be derived from acrylic acid (CH₂CHCO₂H), although it may in practice be made by polymerising methyl acrylate to form —CH₂—CH(CO₂Me)- units and hydrolysis of these units. It may of course alternatively be made from acrylic acid by polymerisation thereof.

Compatibility of the micelles with a hydrophobic environment, and encapsulation capabilities of the micelles for hydrophilic biological molecules may depend on the HLB (hydrophilic-lipophilic balance) of the amphiphilic copolymer. This may be controlled by controlling the hydrophobicity of the hydrophobic endgroup. The hydrophobicity may be adjusted by adjusting the nature of the endgroup and/or its chain length/formula weight. Thus for example a fluorinated endgroup may be more hydrophobic than the corresponding non-fluorinated endgroup. Also, for example, a linear alkyl group will in general increase in hydrophobicity with increasing chain length. The HLB may also be controlled by controlling the hydrophilicity of the monomer units. This may be adjusted by adjusting the nature of the monomer units (for example monomer units derived from acrylic acid will be more hydrophilic than those derived from an co-alkenoic acid). The ratio of different monomer units may also have an influence on the HLB value. Further, the molecular weight of the copolymer will affect the HLB value, by affecting the number of hydrophilic monomer units relative to the number of hydrophobic endgroups per molecule (since the number of endgroups per molecule is limited). In the case where the hydrophobic endgroup comprises an alkyl group, the ratio of hydrophilic monomer units to carbon atoms in the alky group may be between about 2:1 and about 20:1, or about 2:1 and 10:1, 2:1 and 5:1, 5:1 and 20:1, 10:1 and 20:1, 5:1 and 10:1 or 3:1 and 8:1, e.g. about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1 or 20:1 on a mole basis. The HLB, or one or more factors affecting it, may affect the lower critical solution temperature of the polymer. It (they) may affect the temperature at which the micelles lose stability.

The first monomer is such that the copolymer is thermally responsive. It may be an amphiphilic monomer, having hydrophilic and hydrophobic regions. It may be a monomer which may exist in a hydrated state below a transition temperature and in a less hydrated state, or unhydrated state, above the transition temperature. It may be such that monomer units derived therefrom in a polymer or copolymer may exist in a hydrated state is below a transition temperature and in a less hydrated state, or unhydrated state, above the transition temperature. The conversion from hydrated to less hydrated state or unhydrated stage may alter the hydrophilicity of the copolymer. It may alter the conformation of the copolymer. It may alter the self-assembly properties of the copolymer. The conversion from hydrated to less hydrated state or unhydrated state may convert the copolymer from a condition in which it can form reverse micelles to a state in which it is incapable, or less capable, of forming reversed micelles. The first monomer may be an acrylamide or a methacrylamide (optionally substituted on the methyl group). It may be an N-substituted acrylamide. The N-substitution may be an alkyl group or an aryl group, each being optionally substituted. The first monomer may be an N-alkylacrylamide. The alkyl group may be a C1 to C10 straight chain alkyl group (or C1 to C6, C2 to C10, C6 to C10 or C2 to C6, e.g. C1, C2, C3, C4, C5, C6, C7, C8, C9 or C10) or a C3 to C10 branched chain or cyclic alkyl group (or C3 to C8, C3 to C6, C6 to C10 or C4 to C8, e.g. C3, C4, C5, C6, C7, C8, C9 or C10). The length, branching etc. of the N-substituent may affect the transition temperature described above. The first monomer may be N-isopropylacrylamide. It may be a mixture of any two or more of the aforesaid options for first monomer.

The second monomer comprises a carboxylic acid or carboxylate group. It may be a monocarboxylic acid or a salt thereof. It may be a dicarboxylic acid or a salt or acid salt thereof. Suitable monocarboxylic acids include acrylic acid, methacrylic acid, hydroxymethacrylic acid, 1-propenoic acid, 2-propenoic acid etc. Suitable dicarboxylic acids include fumaric acid, maleic acid, pent-2-ene-1,5-dioic acid etc. The second monomer may be a mixture of any two or more of the above or of other suitable carboxylic acid or carboxylate monomers.

The amphiphilic copolymer may be substantially linear. In this case the amphiphilic copolymer may have between about 1 and about 2 hydrophobic endgroups per molecule (recognising that this will be an averaged value due to different chain lengths of copolymer). It may have about 1 to 1.5, 1.5 to 2, 1 to 1.2, 1.2 to 1.5, 1.5 to 1.8 or 1.8 to 2 hydrophobic endgroups per molecule, e.g. about 1, 1.1, 1.2, 1.3, 1.4, 1.5, 1.6, 1.7, 1.8, 1.9 or 2. In the event that the amphiphilic copolymer is substantially branched, there may be cases in which the copolymer has more than 2 hydrophobic endgroups. The hydrophobic endgroup may be sufficiently long and/or hydrophobic that the amphiphilic copolymer can form inverse micelles having a core-shell structure wherein the hydrophobic endgroup forms, or is located in, the shell. The hydrophobic group may be an aryl group, it may be a polyaryl group or a fused aryl group e.g. a biphenyl or terphenyl group, or a naphthyl, anthracyl, phenanthryl or other aryl group. It may be an alkyl group. It may be a C1 to C24 straight chain alkyl group. It may be a straight chain alkyl group with 1 to 18, 1 to 12, 1 to 6, 6 to 24, 12 to 24, 18 to 24, 12 to 18, 14 to 20 or 16 to 20 carbon atoms, e.g. 1, 2, 3, 4, 5, 6, 7, 8, 9, 10, 11, 12, 13, 14, 15, 16, 17, 18, 19, 20, 21, 22, 23 or 24 carbon atoms. It may be a C3 to C24 branched chain or cyclic alkyl group. It may be a branched chain or cyclic alkyl group with 3 to 18, 3 to 12, 3 to 6, 6 to 24, 12 to 24, 18 to 24, 6 to 12, 12 to 18 or 12 to 20 carbon atoms, e.g. 3, 4, 5, 6, 7, 8, 9, 10, 11, 12, 13, 14, 15, 16, 17, 18, 19, 20, 21, 22, 23 or 24 carbon atoms. It may be an undecyl, tetradecyl or octadecyl group. It may comprise a combination of any two or more of aryl, polyaryl, linear alkyl, branched alkyl and cycloalkyl groups. It will be understood that commonly longer chain alkyl groups are obtained from natural sources and are often not pure. Thus when reference is made to a particular chain length of (or number of carbon atoms in) an alkyl group, only that chain length (or number) may be present, or alternatively a distribution of chain lengths (or numbers) may be present centred around that particular value. Thus for example reference to an octadecyl group may include a distribution of C16 to C20 chains in which the most common chain length is C18.

The hydrophobic endgroup may be coupled to one of the monomer units by a suitable linker group. This may be for example an alkyl group, a hydroxyalkyl group, a cycloalkyl group, a triazine ring or other suitable linker group. In one example the endgroup may be coupled via a —S(CH₂)ₙO— group. In this case n may be between 2 and about 24, or about 2 to 18, 2 to 12, 2 to 6, 6 to 24, 12 to 24, 6 to 12 or 4 to 8, e.g. 2, 3, 4, 5, 6, 7, 8, 9, 10, 11, 12, 13, 14, 15, 16, 17, 18, 19, 20, 21, 22, 23 or 24.

The amphiphilic copolymer may have a molecular weight between about 5 and about 20 kDa, or about 5 to 10, 10 to 20 or 10 to 15 kDa, e.g. about 5, 6, 7, 8, 9, 10, 11, 12, 13, 14, 15, 16, 17, 18, 19 or 20 kDa. It may have a narrow molecular weight distribution or it may have a broad molecular weight distribution. It may have a polydispersity (weight average molecular weight/number average molecular weight) of between about 1 and about 10, or about 1 to 5, 1 to 2, 1 to 1.5, 1 to 1.2, 1.5 to 10, 2 to 10, 3 to 10, 5 to 10, 1.5 to 5, 1.5 to 2, 2 to 5 or 2 to 3, e.g. about 1, 1.1, 1.2, 1.3, 1.4, 1.5, 1.6, 1.7, 1.8, 1.9, 2, 2.5, 3, 3.5, 4, 4.5, 5, 6, 7, 8, 9 or 10. It may have a critical micelle concentration in isooctane/hexane/1-propanol (1:0.111:0.123 by volume) of between about 10 and about 200 micromol/L, or about 10 to 100, 10 to 50, 10 to 20, 20 to 200, 50 to 200, 100 to 200, 20 to 100 or 50 to 100 micromol/L, e.g. about 10, 20, 30, 40, 50, 60, 70, 80, 90, 100, 110, 120, 130, 140, 150, 160, 170, 180, 190 or 200 micromol/L. It may have a lower critical solution temperature in the above solvent mixture of between about 30 and about 50° C., or about 30 to 40, 40 to 50, 35 to 45 or 35 to 40° C., e.g. about 30, 31, 32, 33, 34, 35, 36, 37, 38, 39, 40, 41, 42, 43, 44, 45, 46, 47, 48, 49 or 50° C.

The amphiphilic copolymer is capable of forming a micellar solution. The term “micellar solution” in the present specification refers to a system in which micelles are dispersed in a liquid. The micelles are aggregates of a micellised substance (in the present case the amphiphilic copolymer) and may have one or more other materials (e.g. water, biological substance) located therein. The micellar solution may be considered to contain two phases—a dispersed phase (the micelles) and a continuous phase (the liquid). Thus the micellar solution comprises micelles of the amphiphilic copolymer in a liquid. The proportion of the copolymer in the micellar solution may depend in part on the CMC (critical micelle concentration) of the copolymer in the liquid. This will vary depending on the nature of the copolymer and of the liquid. The proportion, or concentration, may for example be between about 1 and about 100 g/L, or between about 1 and 50, 1 and 20, 1 and 10, 1 and 5, 5 and 100, 10 and 100, 20 and 100, 50 and 100, 10 and 80, 10 and 50 or 50 and 80 g/L, e.g. about 1, 2, 3, 4, 5, 6, 7, 8, 9, 10, 15, 20, 25, 30, 35, 40, 45, 50, 55, 60, 65, 70, 75, 80, 85, 90, 95 or 100 g/L.

The size (i.e. diameter) of the micelles may depend on the nature and molecular weight of the amphiphilic copolymer, the nature and quantity of any substances (e.g. biological substances, liquids etc.) encapsulated in the micelles etc. The diameter may be between about 100 and 1500 nm, or about 100 to 1000, 100 to 800, 100 to 500, 100 to 200, 200 to 1500, 500 to 1500, 1000 to 1500, 200 to 1000, 200 to 500 or 500 to 1000 nm, e.g. about 100, 150, 200, 250, 300, 350, 400, 450, 500, 550, 600, 650, 700, 750, 800, 850, 900, 950, 1000, 1100, 1200, 1300, 1400 or 1500 nm. In the context of the present specification, reference to a substance (e.g. biological substance, liquid, salt etc.) “encapsulated” in the micelles does not necessarily indicate that the substance exists in the micelles in one or more discrete regions and may be taken to indicate that the substance is located in the micelles. An encapsulated substance may be distributed homogeneously or heterogeneously through the core of the micelles. It may exist in one or more discrete regions within the micelles or may not exist in discrete regions within the micelles. It may be immobilised in the micelles, in the sense that it is incapable of migrating out of the micelles (unless the micelles are disrupted, as described herein).

The molecules of the amphiphilic copolymer may spontaneously self-assemble into micelles when combined with an appropriate liquid. The micelles may be reverse (or reversed or inverse) micelles. They may have a core-shell structure. The core-shell structure may have a hydrophobic shell surrounding a hydrophilic core. As noted earlier, the amphiphilic copolymer contains at least one hydrophobic region and at least one hydrophilic region. In the micelles, the hydrophobic region(s) may be located in and/or form the shell. In particular the hydrophobic endgroups may be located in the shell. The hydrophilic regions may be located in the core. The monomer units derived from the second monomer may be located in the core. These may be polar due to the carboxylic acid or carboxylate groups thereon.

The liquid may be a solvent. It may be an organic liquid. It may be a non-polar organic liquid. It may comprise a mixture of two or more solvents. It may comprise both polar and non-polar liquids. It may comprise polar and non-polar liquids in proportions such that the liquid is non-polar. It will be understood that all liquids have some degree of polarity, and that reference to a non-polar liquid should be taken to refer to a liquid of low polarity. Suitable non-polar liquids include hydrocarbons or hydrocarbon mixtures. Hydrocarbons such as hexane, heptane, octane, nonane, decane, undecane, dodecane, tetradecane, cyclohexane, cycloheptane, isooctane or other liquid hydrocarbons or mixtures thereof may be used. The mixtures may comprise polar liquids such as alcohols, ethers, ketones, esters and mixtures thereof which are miscible with the non-polar liquid to the extent that they are used in the mixture. A suitable liquid includes isooctane/hexane/1-propanol (1:0.111:0.123 by volume) mixture.

As noted above, the micelles may comprise a core-shell structure. There may be a biological substance located in the core of the micelles. The biological substance may be an enzyme, a protein, a peptide (e.g. an oligopeptide, a synthetic or natural polypeptide, an amino acid), a saccharide, an antibody, an antibody fragment such as an Fab or an Fc or a mixture of these. It may comprise a drug. The biological substance may be catalytically active. Encapsulation within the core of the micelles may protect the biological substance from degradation, denaturation, inactivation or attack due to environmental components which are incapable of penetrating to the core of the micelle. Thus for example, the activity of an encapsulated biological substance (e.g. enzyme) may decrease over 24 hours when located in micelles of a micellar solution according to the invention by less than about 50% after about 12 hours, or less than about 40, 30, 20, 10, 5, 2 or 1%. It may decrease by less than about 50% after about 24 hours, or less than about is 40, 30, 20 or 10%. The core of the micelles may also comprise other components, for example an aqueous liquid such as water, salts etc. The biological substance may be present in the micellar solution at a concentration of between about 10 and about 200 mg/L, or about 10 to 100, 10 to 50, 10 to 20, 20 to 200, 50 to 200, 100 to 200, 20 to 100 or 50 to 100 mg/L, e.g. about 10, 20, 30, 40, 50, 60, 70, 80, 90, 100, 110, 120, 130, 140, 150, 160, 170, 180, 190 or 200 mg/L. The ratio of biological substance to amphiphilic polymer may be between about 0.1 to about 1% by weight, or about 0.1 to 0.5, 0.1 to 0.2, 0.2 to 1, 0.5 to 1, 0.2 to 0.8 or 0.3 to 0.7%, e.g. about 0.1, 0.2, 0.3, 0.4, 0.5, 0.6, 0.7, 0.8, 0.9 or 1% by weight. If present, the aqueous liquid may be present in the micellar solution at about 0.1 to about 0.5% w/v or w/w, or about 0.1 to 0.3, 0.2 to 0.5 or 0.2 to 0.4%, e.g. about 0.1, 0.2, 0.3, 0.4 or 0.5%.

The activity of the biological substance may be enhanced when encapsulated in micelles of the amphiphilic copolymer relative to when they are not encapsulated. This may be particularly pronounced when a liquid is used that is aggressive towards the biological substance. The activity (e.g. biological activity, catalytic activity, enzymatic activity) of the biological substance in the micelles relative to the activity when not in the micelles, both being in the same liquid (i.e. activity of encapsulated substance divided by activity of unencapsulated or naked substance) may be between about 2 and about 100, or about 2 to 50, 2 to 20, 2 to 10, 5 to 100, 10 to 100, 20 to 100, 50 to 100, 5 to 50, 5 to 20, 10 to 50 or 20 to 50, e.g. about 2, 3, 4, 5, 6, 7, 8, 9, 10, 15, 20, 25, 30, 35, 40, 45, 50, 60, 70, 80, 90 or 100.

The amphiphilic copolymer of the present invention may be made by coupling a precursor copolymer to an endcapping reagent so as to attach a hydrophobic endgroup to the precursor polymer to form the amphiphilic copolymer. The precursor copolymer is a copolymer of the first monomer and the second monomer as described above and the endcapping reagent comprises the hydrophobic endgroup. The first and second monomers may be polymerisable by a free radical process. They may be olefinic (e.g. acrylic, styrenic, vinyl ether etc.) monomers.

The precursor copolymer may be made by copolymerising the first monomer and the second monomer. The copolymerisation may be by a free radical polymerisation. It may be initiated by radiation (e.g. uv, gamma ray, electron beam or other radiation) or thermally, or may be spontaneously initiated.

The proportion of the second monomer in the total monomers (and consequently the proportion of monomer units in the precursor polymer and in the amphiphilic polymer that are second monomer units) may be between about 0.1 and about 10% on a weight or mole basis, or about 0.1 to 5, 0.1 to 2, 0.1 to 1, 0.1 to 0.5, 0.5 to 10, 1 to 10, 2 to 10, 5 to 10, 0.5 to 5, 0.5 to 2, 0.5 to 1 or 1 to 2, e.g. about 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9 or 10%, or may be more than 10%.

The copolymerisation reaction may be initiated by means of an initiator. The initiator may be a UV initiator or activator, or may be a thermal initiator. In the case of a thermal initiator, the copolymerisation may comprise heating a mixture of the monomers and the initiator, optionally in a solvent, to a temperature at which the initiator decomposes at a suitable rate for polymerisation of the monomers. This temperature will depend on the nature of the initiator, and the dependence on half-life on temperature for different thermal initiators is well known. Suitable thermal initiators include azo initiators (e.g. AIBN), peroxides (e.g. benzoyl peroxide), hydroperoxides (e.g. cumene hydroperoxide), peroxidicarbonates etc. Suitable UV initiators or sensitisers include benzoin ethers, benzophenones etc. and other well known substances. The initiator may be used in a ratio to total monomer of between about 0.05 and about 1% on a weight or mole basis, or about 0.1 to 1, 0.5 to 1, 0.05 to 0.5, 0.05 to 0.02, 0.05 to 0.1, 0.1 to 0.5, 0.1 to 0.3 or 0.2 to 0.5%, e.g. about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95 or 1% or may be more than 1%. In some cases, the initiator may comprise a functional group which is capable of reacting with the endcapping reagent. In this case, the initiator produces initiator fragments which are incorporated into the precursor copolymer during the copolymerisation reaction. These initiator fragments contain the functional group and can be used to incorporate the endgroup into the amphiphilic copolymer by reaction with the endcapping reagent. The reaction may be conducted at any suitable temperature (depending as described above on the nature of the initiator and/or initiating radiation), e.g. about 20 to about 100° C., or about 20 to 80, 20 to 60, 20 to 40, 40 to 100, 60 to 100 or 40 to 80° C., e.g. about 20, 30, 40, 50, 60, 70, 80, 90 or 100° C. or may be more than 100° C. The reaction time will depend on the temperature, the nature of the initiation and of the monomers, and may for example be between about 0.5 and about 24 hours, or about 1 to 24, 6 to 24, 12 to 24, 0.5 to 12, 0.5 to 6, 0.5 to 2, 1 to 12, 1 to 6, 1 to 3 or 6 to 12 hours, e.g. about 0.5, 1, 1.5, 2, 2.5, 3, 3.5, 4, 4.5, 5, 5.5, 6, 7, 8, 9, 10, 11, 12, 13, 14, 15, 16, 17, 18, 19, 20, 21, 22, 23 or 24 hours. It may be conducted in solution in a solvent that is capable of dissolving the monomers and other reagents. Suitable solvents include toluene, THF, acetone, methyl ethyl ketone diethyl ether, propylene glycol, benzene, tetrahydropyran etc. As is commonly the case for free radical polymerisation reactions, the reaction may be done under reduced oxygen, preferably in the absence of oxygen, as oxygen is a known inhibitor of free radical reactions. The reaction mixture may therefore be degassed prior to commencing the copolymerisation reaction. This may be achieved by bubbling an inert gas having very low oxygen concentration through the reaction mixture. Suitable gases include nitrogen, helium, neon and argon. Alternatively or additionally the reaction mixture may be degassed using one or more (preferably 2, 3, 4 or 5) freeze-pump-thaw cycles.

The step of copolymerising may be conducted in the presence of a chain transfer agent. Suitable chain transfer reagents include mercaptans, certain halides etc. These serve to limit the molecular weight of the precursor copolymer (and hence of the resulting amphiphilic copolymer) and the desired molecular weight may be obtained by balancing the nature of the monomers, the nature and concentration of the chain transfer agent using known methods. In some embodiments, the chain transfer agent comprises a functional group capable of coupling to the endcapping reagent. It may for example comprise a hydroxyl group. It may therefore be a bifunctional chain transfer agent, having a chain transfer functional group and a coupling functional group. The coupling functional group may then serve as an endgroup for the precursor copolymer. Suitable chain transfer agents include mercaptol-alcohols. These include compounds of structure HS(CH₂)ₙOH group, where n may be between 2 and about 24, or about 2 to 18, 2 to 12, 2 to 6, 6 to 24, 12 to 24, 6 to 12 or 4 to 8, e.g. 2, 3, 4, 5, 6, 7, 8, 9, 10, 11, 12, 13, 14, 15, 16, 17, 18, 19, 20, 21, 22, 23 or 24. Other suitable compounds include mercaptophenols such as meta- or para-HSC₆H₄OH. A suitable concentration of chain transfer agent relative to monomer is for example between about 0.1 and about 10% on a weight or mole basis, or about 0.1 to 5, 0.1 to 2, 0.1 to 1, 0.1 to 0.5, 0.5 to 10, 1 to 10, 2 to 10, 5 to 10, 0.5 to 5, 0.5 to 2, 0.5 to 1 or 1 to 2, e.g. about 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9 or 10%, or may be more than 10%.

The step of coupling the precursor copolymer to the endcapping reagent comprises reacting the precursor copolymer with the endcapping reagent. Thus a functional group on the precursor copolymer (the coupling functional group described above) may be reacted with a functional group on the endcapping reagent. Numerous suitable chemistries for such coupling are known. One suitable chemistry is the reaction of an alcohol with a halide. Thus an OH group on the precursor copolymer may be reacted with a halide group on the encapping reagent to attach the endgroup in the encapping reagent to the precursor copolymer. Suitable endgroups have been described earlier, and consequently suitable endcapping reagents include the corresponding halides (for example chlorides, bromides or iodides) e.g. alkyl halides. Other suitable endcapping reagents include arylmethyl halides such as benzyl chloride, benzyl bromide, naphthylmethyl bromide etc. Other coupling reactions that are well known in the art may also be used. These may comprise any of the well known methods of introducing chemical groups into a molecule. These include “click” chemistry. Suitable click chemistry may include for example cycloaddition reactions, such as the Huisgen 1,3-dipolar cycloaddition, Cu(I) catalyzed azide-acetylene cycloaddition, Diels-Alder reaction, nucleophilic substitution to small strained rings (e.g. epoxy and aziridine rings), formation of ureas and amides and addition reactions to double bonds, e.g. epoxidation, dihydroxylation.

A micellar solution of the amphiphilic copolymer of the invention may be made by combining the amphiphilic copolymer and an organic liquid so as to form micelles of the amphiphilic copolymer in the liquid. The polymer may be added at a ratio of between about 1 and about 100 g/L of liquid, as described above. The nature of the liquid has also been described earlier. The step of combining may comprise stirring, swirling, shaking, mixing, sonicating or otherwise agitating the combined copolymer and liquid so as to form the micelles.

As noted above, the micelles may contain a biological substance, e.g. an enzyme or some other type of biological substance. In order to produce micelles of the copolymer which contain the biological substance, the amphiphilic copolymer and an organic liquid may be combined with the biological substance, optionally in a second liquid.

The second liquid may be a solvent for the biological substance. The biological substance may be added as a solution in the second liquid, or it may be added as a suspension in the second liquid or as an emulsion in the second liquid or as a microemulsion in the second liquid or as a dispersion in the second liquid. The second liquid may be an aqueous liquid. It may comprise water. It may additionally comprise other components, for example salts, buffers etc. The second liquid may be at a suitable pH for the biological substance, for example at a suitable pH for optimal, or at least acceptable, activity of the biological substance. It may be buffered to the suitable pH. The suitable pH will depend on the nature of the biological substance. It may be between about 6 and about 8, or about 6 to 7, 7 to 8, 6.5 to 7.5 or 7 to 7.5, e.g. about 6, 6, 6.5, 7, 7.5 or 8. The second liquid may for example be PBS (phosphate buffered saline). The second liquid may be immiscible with the organic liquid. It may in some cases be miscible or partially miscible therewith. In some embodiments, the second liquid is aqueous and the organic liquid is substantially non-polar, whereby the two liquids have low miscibility with each other.

The micellar solution may be made by combining (optionally agitating) the amphiphilic copolymer and the organic liquid so as to form micelles of the copolymer in the liquid, whereby the micelles adopt a core-shell structure in which the hydrophobic endgroups are located in the shell and the monomer units derived from the second monomer are located in the core. The amphiphilic may spontaneously self-assemble to form the micelles. A solution of a biological substance in the second liquid may then be added to the organic liquid (i.e. to the resulting micellar solution of the copolymer in the organic liquid) so as to form the micellar solution wherein the biological substance is located in the core of the micelles. It may be desirable or necessary to agitate the mixture in order to facilitate entry of the biological substance into the micelles. This may comprise stirring, swirling, shaking, mixing, sonicating or otherwise agitating said mixture. The second liquid may also enter the micelles and be located therein. The second liquid in the micelles, if present, may at least partially solvate the biological substance. This may improve the stability of the biological substance. It may also provide suitable conditions, e.g. of pH, inside the micelles for activity of the biological substance.

In an alternative process, the organic liquid, the amphiphilic copolymer and the biological substance, optionally in the second liquid, may be combined, and then agitated, whereby micelles of the copolymer in the organic liquid form and contain the biological substance, and optionally also contain at least some of the second liquid.

In some embodiments of the processes for making the micelles, the second liquid may be absent. Thus the above processes may be performed as described but in the absence of the second liquid. This may particularly suitable in cases in which the biological substance is a liquid at the temperature at which the micellar solution is formed. It will be readily understood that the above processes for formation of a micellar solution should be conducted at a temperature below the lower critical solution temperature (LCST) of the amphiphilic copolymer.

If a biological substance is used in producing the micellar solution the ratio of biological substance to amphiphilic polymer may be between about 0.1 to about 0.5% as described earlier. As noted, it may be added in a second liquid (e.g. in solution therein). The concentration of the biological substance in the second liquid may be between about 10 and about 50 mg/ml, or about 10 to 40, 10 to 30, 10 to 20, 20 to 50, 30 to 50 or 20 to 40 mg/ml, e.g. about 10, 15, 20, 25, 30, 35, 40, 45 or 50. The ratio of biological substance in second liquid to polymer in organic liquid may be between about 0.1 and about 0.5% by volume or by weight, or about 0.1 to 0.3, 0.2 to 0.5 or 0.2 to 0.4%, e.g. about 0.1, 0.2, 0.3, 0.4 or 0.5%. The ratio of the second liquid to the amphiphilic copolymer may be between about 10 to about 200 on a mole basis, or about 10 to 100, 10 to 50, 20 to 200, 50 to 200, 100 to 100, 20 to 150, 30 to 150, 100 to 150 or 30 to 100, e.g. about 10, 20, 30, 40, 50, 60, 70, 80, 90, 100, 110, 120, 130, 140, 150, 160, 170, 180, 190 or 200.

The biological substance may be readily separated from a micellar solution according to the invention in which the biological substance is located within the micelles of the micellar solution. This may be achieved by heating the micellar solution to a temperature above the lower critical solution temperature (LCST) of the amphiphilic copolymer. As noted, the LCST of the copolymer may be between about 30 and about 50° C. When the LCST is exceeded, the micelles at least partially dissociate, thereby releasing the biological substance. It may then be isolated using well known separation techniques. The biological substance may be precipitated from the liquid following heating to a temperature above the LCST. In many instances it may be preferable that the heating be to a temperature below the denaturation temperature, or decomposition temperature or degradation temperature, of the biological substance in order to prevent damage to the biological substance in the process. Thus the maximum temperature to which the micellar solution should be heated will vary with the nature, particularly the stability, of the biological substance. Such temperatures are generally well documented.

A micellar solution according to the present invention in which a biological substance is located in the micelles of the micellar solution may be used for conducting a reaction of at least one reagent to produce a product when the biological substance is capable of catalysing the reaction. Thus the at least one reagent is combined with the micellar solution. It or they may be soluble in the micellar solution, in particular in the continuous phase of the micellar solution. In many embodiments of this, the biological substance is an enzyme, and the reaction is an enzyme catalysed reaction. Many such reactions are known, for example an esterification reaction. A suitable reaction is the esterification of lauric acid and 1-propanol to produce 1-propyl laurate, catalysed by Candida rugosa lipase. In this case, the lipase is the biological substance, which is located in the micelles, and the reagents are 1-propanol and lauric acid which are suitably located in the continuous organic phase of the micellar solution. The reaction should be conducted at a temperature below the LCST of the amphiphilic copolymer, so as to retain the integrity of the micelles.

In reactions as described above, it is thought that the reagent(s) diffuse through the shell of the micelles to the core. In the core, it (they) reacts to generate the product by way of a reaction involving (in many cases catalysed by) the biological substance. The product then diffuses out of the micelles through the shell and into the continuous phase of the micellar solution.

The additional step of separating the biological substance from the micellar solution may be conducted. Suitably this step may comprise heating the micellar solution to a temperature above the lower critical solution temperature of the copolymer. This step is preferably conducted after at least some of the at least one reagent has been reacted to produce the product. Thus following at least partial conversion of reagent(s) to product, the biological substance may be released from the micelles into the continuous phase of the micellar solution. As noted earlier, this continuous phase may be detrimental to the biological substance. For example, the biological substance may be an enzyme and the continuous phase may be substantially hydrophobic, and therefore exposure to the continuous phase may cause denaturation of the enzyme. In this manner the reaction may be stopped at any desirable time simply by raising the temperature of the micellar solution to a temperature above the lower critical solution temperature of the copolymer, as this leads as described above to denaturation of the enzyme.

### BRIEF DESCRIPTION OF THE DRAWINGS

A preferred embodiment of the present invention will now be described, by way of an example only, with reference to the accompanying drawings wherein:

FIG. 1 shows a diagrammatic representation of a micelle in a micellar solution;

FIG. 2 is a ¹H NMR (nuclear magnetic resonance) spectrum of P(NIPAAm-co-AA)-b-C₁₈H₃₇;

FIG. 3 shows a plot of transmittance of polymer solution (PBS, pH 7.4 and 5 mg/mL) as a function of temperature at 500 nm;

FIG. 4 shows a plot of peak intensity at 336 nm as a function of log C for Polymer III in the mixed solvent, isooctane/hexane/1-propanol (1:0.111:0.123 in volume);

FIG. 5 shows a typical TEM (transition electron microscope) image of enzyme-loaded reversed Polymer III micelles;

FIG. 6 is a graph showing the effect of pH on catalytic activity of immobilized lipase (polymer concentration=24 mg/mL, enzyme concentration=25 mg/mL PBS, W₀=83.3);

FIG. 7 is a graph showing the effect of polymer concentration on catalytic activity of immobilized lipase (enzyme concentration=25 mg/mL PBS, pH 7.4, W₀=83.3); and

FIG. 8 is a graph showing the effect of lipase concentration on catalytic activity of immobilized lipase (polymer concentration=12 mg/mL, PBS, pH 7.4, W₀=83.3).

### DETAILED DESCRIPTION OF THE PREFERRED EMBODIMENTS

The inventors have used thermally responsive reversed polymer micelles to immobilize enzymes in order to overcome the problems associated with the presence of high concentrations of low molecular mass surfactants associated with conventional micelle systems.

Poly(N-isopropylacrylamide) (PNIPAAm) or its copolymers are of particular interest due to their thermal responsiveness. PNIPAAm exhibits a lower critical solution temperature (LCST) of about 32° C. in aqueous solutions, below which the polymer is water soluble and above which it becomes water insoluble. As such, the micelles self-assembled from hydrophobically modified PNIPAAm copolymers are stable below the LCST, but deform at temperatures higher than the LCST because of the loss of the hydrophobicity/hydrophilicity balance of the core-shell structure and thereby release the enclosed compounds. Copolymerization with a more hydrophilic or hydrophobic monomer can increase or decrease the LCST of PNIPAAm. PNIPAAm-based amphiphilic copolymers have been widely investigated to form micelles in aqueous solutions for biomedical applications. Poly(N-isopropylacrylamide-co-N,N-dimethylacrylamide-co-10-undecenoic acid), poly(N-isopropylacrylamide-co-N,N-dimethylacrylamide)-b-poly(lactide-co-glycolide), cholesteryl end-capped poly(N-isopropylacrylamide-co-N,N-dimethylacrylamide) and cholesteryl grafted poly[N-isopropylacrylamide-co-N-(hydroxymethyl)acrylamide] polymers have been synthesized and utilized to form micelles for incorporation of anticancer drugs. The controlled release of the enclosed drugs at target tissues can be achieved by local heating.

In one embodiment the present invention relates to synthesis of alkyl end-capped poly(N-isopropylacrylamide-co-acrylic acid) (P(NIPAAm-co-AA)) and its fabrication by self-assembly into thermally responsive reversed micelles. These reversed micelles have been successfully employed for the immobilization of enzymes. Alkyl groups were chosen as the shell-forming segment of the polymer because such groups are compatible with, or may be dissolved in, many nonpolar solvents such as hexane and isooctane, which are often employed for the synthesis of chiral pharmaceuticals. Acrylic acid (AA) was introduced into the core-forming block of the polymer as it can increase the LCST (lower critical solution temperature) of the polymer to a degree at that enzymes encapsulated in the resulting micelles possess high activity. Additionally acrylic acid groups provide negative charges necessary for the formation of reversed micelles.

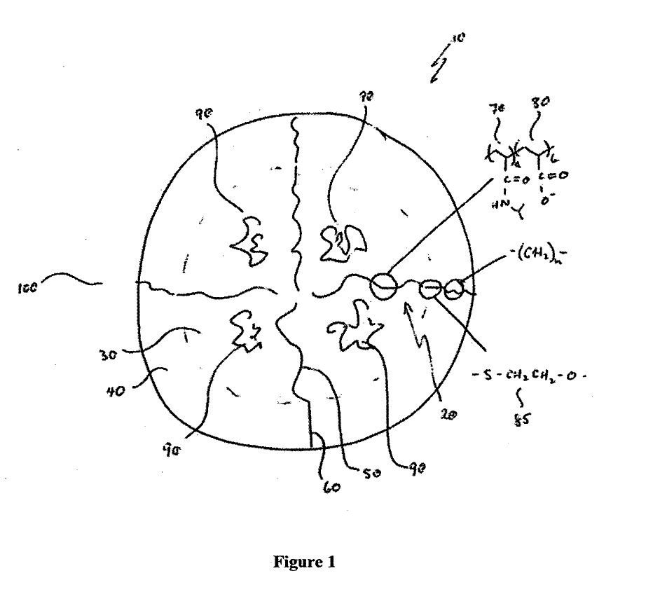

FIG. 1 shows a diagram of a micelle as described above. Thus micelle 10 is formed from self-assembly of amphiphilic copolymer molecules 20. FIG. 1 shows only 4 molecules 20, however in reality more than this would be likely to be present. Micelle 10 has a core-shell structure, in which core 30 is surrounded by shell 40. Each molecule 20 has a hydrophilic region 50, which primarily resides in the core, and a hydrophobic region 60 which resides primarily in the shell. As shown, hydrophilic region 50 comprises monomer units 70 derived from N-isopropylacrylamide, which, below the LCST will be hydrated. Units 70 are such that copolymer molecules 20 are thermally responsive. Hydrophilic region 50 also comprises monomer units 80 (in a ratio to units 70 of about 100:1 units 70: units 80, i.e. a:b is about 100:1). Units 80 are derived from acrylic acid, and are ionised in basic pH environments as shown, and will protonate at an appropriately acidic pH. Hydrophilic region 50 is linked to hydrophobic region 60 by linker group 85, wherein the sulfur atom is coupled to hydrophilic region 50 and the oxygen atom is linked to hydrophobic region 60. Enzyme molecules 90 are located in core 30, as they are hydrophilic. Commonly core 30 also contains other materials (not shown) which enhance the stability of enzyme molecules 90, such as buffers. Micelles 10 are dispersed within hydrophobic liquid 100, which stabilises micelles 10 by providing a hydrophobic environment for hydrophobic groups 50, such that shell 20 shields hydrophilic core 30 from hydrophobic liquid 100.

As noted earlier, a micellar solution containing micelles such as that described above, may be used to convert one or more starting materials to a product when the micelles contain enzymes capable of catalysing that conversion. Thus in use, one or more reagents are added to hydrophobic liquid 100, which contains micelle 10. It will be understood that the micellar solution contains large numbers of micelles, only one of which is shown in FIG. 1. The reagent(s) diffuse from liquid 100 through shell 40 of micelle 10 to core 30. In core 30, the one or more reagents encounter enzyme molecules 90, which are capable of catalysing reaction of the reagent(s) to the product. The ensuing reaction generates the product by way of a reaction catalysed by enzyme molecules 90. The product then diffuses out of micelles 10 through shell 40 and into hydrophobic liquid 100, i.e. into the continuous phase of the micellar solution. The products may then be recovered using standard separation technicques.

In an example, Candida rugosa lipase, a model enzyme was successfully immobilized into the reversed micelles in the isooctane/hexane/1-butanol (1:0.111:0.123 by volume) mixture. The immobilized lipase gave high activity and stability for the esterification of lauric acid and 1-butanol. It showed higher catalytic activity than naked (unimmobilised) enzyme. Moreover, lipase immobilized in these micelles was much more stable than lipase located in conventional sodium bis(2-ethylhexyl) sulfosuccinate micelles. In addition, lipase precipitated from the reaction mixture after heating, indicating that the immobilized enzyme can be recovered from the reaction mixture by simply changing the environmental temperature to a value slightly higher than the LCST of the polymer.

The effects of pH, water content, polymer and enzyme concentration on the catalytic activity of the immobilised enzymes were investigated. The optimized fabrication conditions of lipase-loaded reversed micelles, under which lipase gave the highest activity, were as follows: polymer concentration, 12 mg/mL; enzyme concentration, 25 mg/mL phosphate buffered saline (PBS); pH, 7.4; W₀, 83.3. Lipase immobilized in these micelles was much more stable than that in conventional sodium bis(2-ethylhexyl) sulfosuccinate micelles. More importantly, the size of lipase-immobilized micelles decreased, and the enzyme solution precipitated from the reaction mixture when the temperature increased to a value slightly higher than the LCST of the polymer. This indicates that the enzyme can be recovered, and the reaction can be terminated by simply changing the environmental temperature. These thermally responsive micelles therefore make a promising system for enzyme immobilization.

### EXAMPLES

### Materials

N-Isopropylacrylamide (NIPAAm, Sigma-Aldrich) was purified by re-crystallization from n-hexane. Acrylic acid (Sigma-Aldrich) was purified by vacuum distillation. Tetrahydrofuran (THF, Merck) was dried over sodium. All other chemicals were of analytical grade, and used as received.

Synthesis of alkyl end-capped P(NIPAAm-co-AA)

The copolymer P(NIPAAm-co-AA) was synthesized by radical polymerization of NIPAAm and AA using benzoyl peroxide (BPO) as an initiator and 2-hydroxyethanethiol as a chain transfer agent. N-isopropylacrylamide (11.20 g), acrylic acid (72.06 mg), 2-hydroxyethanethiol (78.13 mg), and BPO (40.37 mg) were dissolved in 100 mL of THF. The solution was degassed by bubbling nitrogen for 20 minutes. The reaction mixture was then refluxed for 8 hours under nitrogen. The product was then precipitated by addition of diethyl ether, and purified by reprecipitation twice from diethyl ether using a slow liquid-liquid diffusion method. The molecular weight of the polymer was determined by gel permeation chromatography (GPC, Waters, polystyrene standards), using THF as the mobile phase (elution rate: 1 mL/min) at 25° C.

Amphiphilic copolymers with different chain length of alkyl group, including Polymer I (—C₁₁H₂₃), Polymer II (—C₁₄H₂₉) and Polymer III (—C₁₈H₃₇), were prepared by SN2 substitution reaction (Scheme 1).

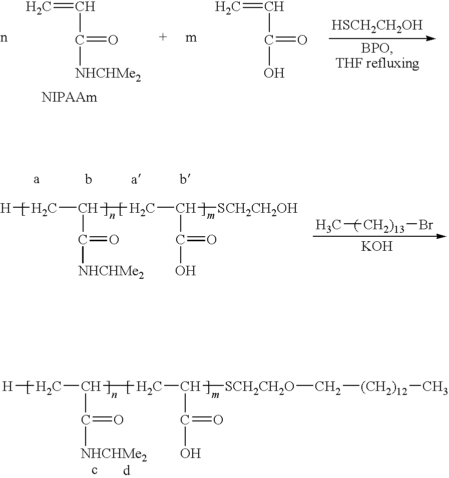

In a typical reaction, potassium hydroxide (3.4 g) was ground to a fine powder and dissolved in 100 mL of THF together with P(NIPAAm-co-AA). The solution was degassed by bubbling nitrogen for 20 minutes. 1-Bromotetradecane (1.25 g) was then dissolved in 20 mL of THF, and added to the mixture. The reaction mixture was then stirred for 2 days under nitrogen. The product was dialyzed against THF using a dialysis membrane with a molecular weight cut-off of 2000 (Spectr/Por) at room temperature for 4 days. The final product was collected after evaporation of THF, and dried in a vacuum oven overnight. The chemical structure of the resulting block polymers was confirmed by ¹H NMR (Bruker Avance 400, 400 MHz) spectroscopy. The lower critical solution temperature (LCST) values of the polymers in PBS (pH 7.4) were determined at the temperatures showing an optical transmittance of 50%. The optical transmittance of the polymers was measured at 500 nm with a UV-Vis spectrometer (Shimadzu, UV-2501PC, Japan). Sample cells were thermostated with a temperature-controller (Shimadzu, TCC-240A, Japan). The heating rate was 10° C./min.

Critical Micellar Concentration (CMC)

The CMC values of Polymer III and AOT in the mixture solvent, isooctane/hexane/1-propanol (1:0.111:0.123 in volume) were determined according to a method described by Subramanian et al. (R. Subramanian, S. Ichikawa, M. Nakajima, T. Kimura, T. Maekawa, Eur. J. Lipid Sci. Technol. 2001, 103, 93). A fixed concentration of polymer or AOT was dissolved in the solvent by mixing overnight. 7, 7, 8, 8-Tetracyanoquinodimethane was added to the solutions at a concentration of 1 mg/ml. The mixtures were shaken for 5 hours at room temperature, and then centrifuged at 1000 rpm for 20 minutes to remove excess 7, 7, 8, 8-tetracyanoquinodimethane (Eppendorf Centrifuge 5417R, Germany). Absorbance of the solutions was recorded on a UV-Vis spectrophotometer (Janco V-570, Japan) from 250 to 500 nm, and the corresponding solvent was used as reference. The intensity of the peak at 336 nm was plotted as a to function of logarithm of polymer concentration. The CMC value was taken from the intersection of the tangent to the curve at the inflection with the horizontal tangent through the points at low concentrations. The measurements were repeated in triplicate, and an average value was used.

Particle Size Analysis

The particle size of freshly prepared micelles was measured by Zetasizer 3000 HAS (Malvern Instrument Ltd., Malvern, UK) equipped with a He—Ne laser beam at 658 nm (scattering angle: 90°). Each measurement was repeated 10 times. An average value was obtained from the ten measurements.

Enzyme Immobilization

Reversed micelles containing Candida rugosa lipase were prepared by direct injection of an aqueous solution of Candida rugosa lipase into the polymer/solvent solution. The lipase was dissolved in PBS at varying pH and concentration, and then centrifuged at 14000 rpm for 5 minutes to remove insoluble impurities. The polymer was dissolved in isooctane/hexane/1-propanol mixture (1:0.111:0.123 in volume) at different concentrations.

To optimize the preparation conditions, the effects of polymer concentration, water content (W₀=[water]/[surfactant]), and lipase concentration on the activity of lipase were examined. Polymer concentration was varied form 12 to 72 mg/mL at W₀ of 83.3 and lipase concentration of 25 mg/mL PBS (pH 7.4). W₀ was changed from 33.3 to 150.0 at polymer concentration of 24 mg/mL. The concentration of lipase in PBS buffer (pH 7.4) was varied from 25 to 300 mg/mL at polymer concentration of 12 mg/mL. Polymer concentration was calculated based on the total volume of the reversed micellar solution.

Assay of Lipase Activity and Stability

The reaction mixture (10 mL) consisted of lauric acid (0.1M), and naked or immobilized lipase. The mixture was incubated at 30° C. for 3 hours with continuous stirring. Reaction samples (1 mL) were withdrawn and mixed with 10 mL of the mixed solvent of ethanol and acetone (1:1 in volume). The unreacted lauric acid was determined by titration with 0.05 M NaOH. The catalytic activity of the enzyme was defined as the amount of acid consumed divided by the amount of lipase used per minute. The stability of lipase was evaluated by analyzing the residual activity at different time intervals at 30° C.

TEM

The morphology of lipase-loaded reversed micelles was observed using a to transmission electron detector (TED) attached to a field-emission scanning electron microscope (FESEM, JEOL7400) and operated at 30 k eV. A drop of the freshly prepared lipase-loaded reversed micelle solution (polymer concentration=12 mg/mL, enzyme concentration=25 mg/mL PBS, W₀=83.3) containing 0.01 (w/v). % phosphotungstic acid was placed on a copper grid coated with formvar (polyvinyl formal) film and thin carbon is film, and was air-dried at room temperature.

### Results and Discussion

Synthesis of Thermally Responsive Amphiphilic Copolymers

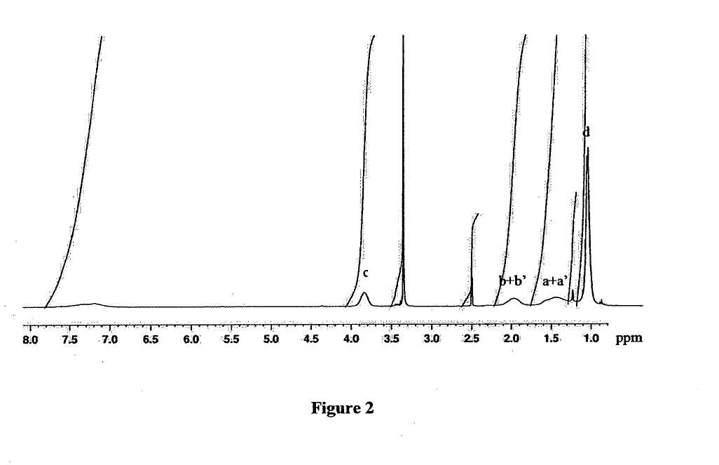

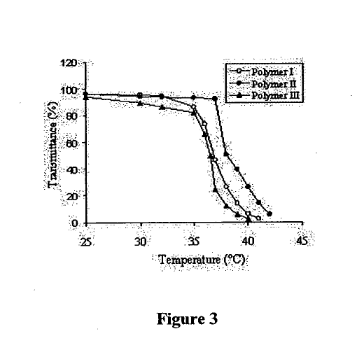

Alkyl end-capped P(NIPAAm-co-AA) amphiphilic copolymers were synthesized in two steps. Hydroxy-terminated P(NIPAAm-co-AA) was first synthesized by radical polymerization using 2-hydroxyethanethiol as a chain transfer agent. The success of the copolymerization of NIPAAm and AA in the presence of the chain transfer agent was evidenced by the absence of vinylic proton signals at δ 5.4-6.6 in the ¹ H NMR spectrum of the polymer (see FIG. 2). The broad peaks at δ 1.2-1.6 (Signal a+a′) and at δ 1.9-2.1 (Signal b+b′) were attributed to the protons of —CH₂— and —CH— groups respectively, in the NIPAAm and AA moieties. Other proton signals from iso-propyl groups (—CHMe₂ at δ 3.84 and —CHMe₂ at δ 1.0, Signals c and d, respectively) were also observed, and their chemical shifts were similar to those of the monomers. The average weight molecular weight of this polymer was about 12 kDa. The content of carboxylic acid groups was determined to be 42.7 mg per gram of polymer by titration with 0.01N NaOH using phenolphthalein as an indicator. The hydrogen in the hydroxyl group of P(NIPAAm-co-AA) was then substituted by bromide of 1-bromotetradecane, 1-bromooctadecane or 1-bromoundecance to form alkyl end-capped P(NIPAAm-co-AA) amphiphilic copolymers. As shown in FIG. 3, the LCST values of Polymer I, Polymer II and Polymer III were similar, being 37.4, 38.2 and 37.7° C. respectively, which were higher than that of PNIPAAm due to the presence of AA molecules. Since the polymers formed core-shell structured micelles in the buffer, their LCST was independent of the core formed from the hydrophobic block but determined by the shell made from the hydrophilic block. All the three copolymers were synthesized based on P(NIPAAm-co-AA) of the same length, resulting in similar LCST values.

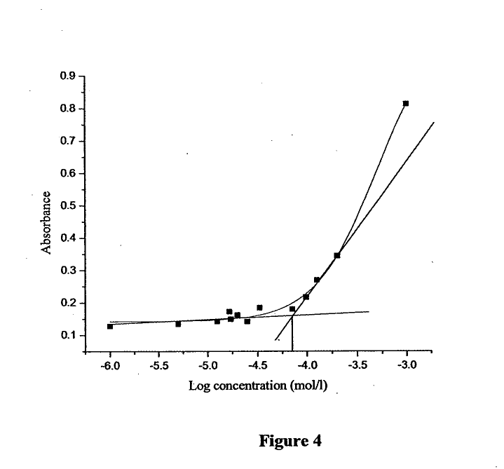

These polymers were readily soluble in polar solvents such as butanol, propanol and chloroform but had limited solubility in nonpolar solvents such as hexane and isooctane. However, the presence of a small amount of polar solvent significantly increased their solubility in nonpolar solvents. For example, they were readily soluble in isooctane/butanol (or propanol or chloroform) and hexane/propanol mixtures, and formed reversed micelles. The critical micellar concentration (CMC) of the polymer in the mixed isooctane/hexane/1-propanol solvent (1:0.111:0.123 by volume) was analyzed in comparison with AOT. FIG. 4 illustrates the CMC of Polymer III. Polymer III formed micelles at a much lower concentration when compared to AOT, and the CMC values of Polymer III and AOT were 7.2×10⁻⁵ mol/l and 4.0×10⁻³ mol/l respectively. This indicates that the amphiphilic copolymer possessed a greater ability to form reversed micelles than the small molecular weight surfactant. Moreover, the reversed micelles formed from the polymers contained a considerable amount of enzyme and the catalytic activity of the enzyme was retained. Table 1 lists the activity of lipase immobilized in the reversed micelles formed from Polymer I, Polymer II and Polymer III respectively, which were tested under the same conditions i.e. solvent isooctane/hexane/1-propanol mixture, 1:0.111:0.123 by volume; polymer concentration 25 mg/mL solvent; enzyme concentration 25 mg/mL PBS buffer (pH 7.4) and W₀ (molar ratio of water to polymer) 83.3.

TABLE 1
Catalytic activity of lipase immobilized in reversed
micelles made from polymers with various lengths of
alkyl (polymer concentration = 24 mg/mL, enzyme
concentration = 25 mg/mL PBS, pH 7.4, W₀ = 83.3).

| Polymer | Polymer I | Polymer II | Polymer III |
|---|---|---|---|
| Catalytic activity [g/g | 1.34 × 10⁻² | 2.24 × 10⁻² | 3.44 × 10⁻² |
| (enzyme) · min] | | | |

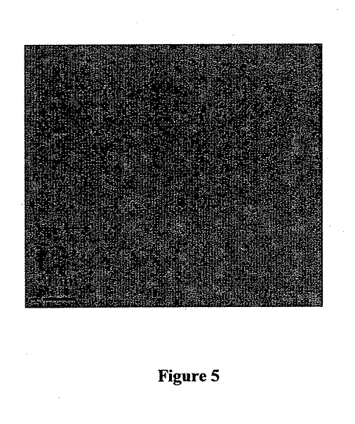

An increased alkyl chain provided greater catalytic activity of lipase. Polymer III micelles yielded the highest activity. The inventors hypothesise that this is because Polymer III with the longest hydrophobic chain produced the most stable micelles in the mixed solvent. Consequently, in the following work, Polymer III was employed. The catalytic activity of lipase immobilized in Polymer III micelles was compared with that of naked lipase. The immobilized lipase gave much higher catalytic activity [1.99×10⁻² g/g (enzyme)·min versus 7.37×10⁻⁴ g/g (enzyme)·min]. This may be because the micelles prevented the enzyme from denaturation by the organic solvents. FIG. 5 shows a TEM picture of lipase-loaded reversed Polymer III micelles, indicating that the micelles were well formed, and were spherical in nature.

### Properties of Enzyme Immobilized in Reversed Micelles

Effect of pH on Catalytic Activity

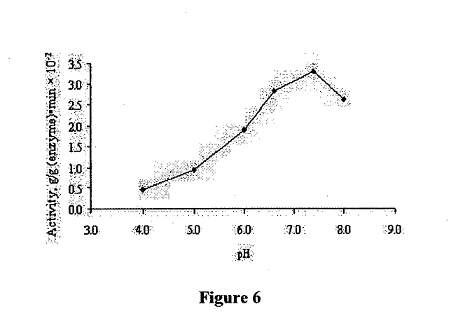

One of the most important factors affecting the catalytic activity of lipase is the pH of the buffer solution because the charge density of enzyme surface changes as a function of pH. As shown in FIG. 6, the catalytic activity of immobilized lipase was very low at low pH such as pH 4.0 and 5.0. Increasing pH led to an improved catalytic activity, and the catalytic activity reached the highest level around pH 7.4, close to the pI value of lipase (7.0). However, further increasing pH resulted in a decreased catalytic activity. At low pH (i.e. pH 4.0 and 5.0), the net charge of the enzyme surface was positive. This led to strong electrostatic interactions between the enzyme molecules and the carboxylic acid groups, especially the protonated carboxylic acid groups (pKa of acrylic acid: 4-4.5) and thus low catalytic activity. However, the net charge of the enzyme surface was minimized around the pI, and the electrostatic interactions were thus the weakest, resulting in the highest catalytic activity.

Effect of Water Content (W₀)

W₀ is another important factor influencing enzyme loading level and catalytic activity. It reflects the hydration degree and the core size of the reversed micelles. Table 2 displays the catalytic activity of lipase as a function of W₀. Similar to other surfactant micelles, it is characterized by a bell-shaped curve.

TABLE 2
Effect of W₀ on catalytic activity and particle size of lipase-loaded micelles (polymer
concentration = 12 mg/mL, enzyme concentration = 25 mg/mL PBS, pH 7.4).

| W₀ | 33.3 | 66.6 | 83.3 | 116.6 | 150.0 |
|---|---|---|---|---|---|
| Catalytic activity [g/g | 2.81 × 10⁻² | 2.81 × 10⁻² | 5.60 × 10⁻² | 3.89 × 10⁻³ | 1.00 × 10⁻³ |
| (enzyme) · min] | | | | | |
| Diameter (nm) | 229 | 532 | 721 | 1933 | — |

The optimum value for W₀ was 83.3, much higher than that of AOT and AOT-modified micelles (W₀=8.0), indicating that higher enzyme loading could be achieved using Polymer III. From Table 2, it can also be seen that the effective diameter of the micelles increased with increasing W₀, and it was 721 nm at the optimum W₀. At W₀ of 150.0, micelles were not stable. It has been suggested that the core size of the reversed micelles is most comparable to the size of the immobilized enzyme at the optimum W₀, providing the highest enzyme activity.

Effect of Polymer Concentration

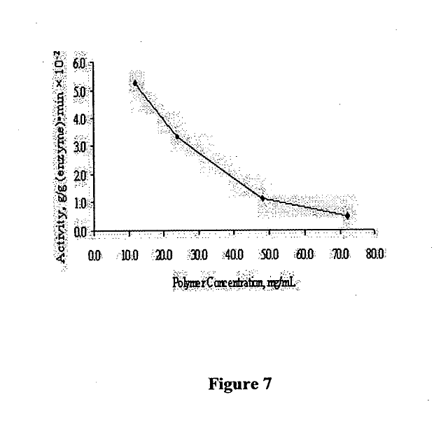

The effect of polymer concentration on the catalytic activity of lipase is shown in FIG. 7. Increasing polymer concentration reduced the activity. It was observed that the size of lipase-loaded micelles increased from 731 to 1510 nm as the polymer concentration increased. It was therefore hypothesised that increased particle size might lead to the increase in the mass-transfer barriers of substrates, decreasing the catalytic activity of lipase. Another possible reason is that larger micelles may not have been stable enough to protect the enzyme from the direct contact with the external organic phase during the enzymatic reaction, leading to the reduction in the catalytic activity.

Effect of Enzyme Concentration in Reversed Micelles

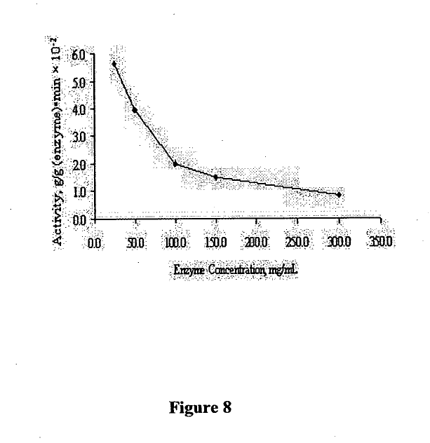

FIG. 8 shows the effect of lipase concentration on the catalytic activity of lipase. An increased enzyme concentration yielded lower catalytic activity. This may be because lipase molecules entangled together at high concentrations, limiting the lipase mobility and its access to the reaction substrates. On the other hand, an increased enzyme concentration yielded the increase in the viscosity of the enzyme solution, which might lead to unstable reversed micelles.

Lipase Stability and Separation

The stability of an enzyme is important to its practical applications. Table 3 shows the stability of lipase immobilized in Polymer III reversed micelles over 24 hours of testing.

TABLE 3
Stability of lipase immobilized in Polymer III micelles (polymer concentration = 24
mg/mL, enzyme concentration = 25 mg/mL PBS, pH 7.4, W₀ = 83.3).

| Time (hours) | 3 | 6 | 9 | 12 | 24 |
|---|---|---|---|---|---|
| Catalytic activity [g/g | 3.30 × 10⁻² | 2.83 × 10⁻² | 3.30 × 10⁻² | 3.30 × 10⁻² | 2.59 × 10⁻² |
| (enzyme) · min] | | | | | |

The catalytic activity of lipase did not change much over 24 hours. In sharp contrast, the activity of lipase immobilized in AOT micelles has been reported to decrease rapidly as a function of time, to lose most of its activity within the first 10 hours. The polymer developed in the present invention formed stable reversed micelles in mixed solvent, which provided a stable microenvironment for enzyme immobilization, protecting the enzyme from degradation against organic solvents.

The possibility of separating the enzyme from the thermally responsive reversed micelles was examined by changing the environmental temperature. The effective diameter of micelles decreased as increasing the temperature to a value higher than the LCST of the polymer. For example, the diameter reduced to 432 nm from 788 nm when the temperature increased from 30 to 40° C. This is considered to be because the hydrophilic block of the polymer became hydrophobic when the temperature increased above the LCST, releasing the lipase-containing buffer solution. Buffer droplets, precipitated from the reaction mixture, were observed on the wall of the beaker. This indicates that the thermosensitivity of the polymer may be exploited to recover the enzyme after the completion of reaction, or to terminate the reaction by simply increasing the temperature slightly higher than the reaction temperature.

1. An amphiphilic copolymer comprising:
monomer units derived from a first monomer;
monomer units derived from a second monomer, said second monomer comprising a carboxylic acid or carboxylate group; and
at least one hydrophobic endgroup;
wherein the first monomer is such that the copolymer is thermally responsive.

2. The copolymer of claim 1 wherein the first monomer is an N-alkylacrylamide.

3. The copolymer of claim 2 wherein the N-alkylacrylamide is N-isopropylacrylamide.

4. The copolymer of claim 1 wherein the second monomer is selected from the group consisting of acrylic acid, methacrylic acid, acrylate and methacrylate.

5. The copolymer of claim 1 wherein the hydrophobic endgroup is a C1 to C24 straight chain alkyl group or a C3 to C24 branched chain alkyl group.

6. The copolymer of claim 5 wherein the hydrophobic endgroup is an octadecyl group.

7. The copolymer of claim 1 wherein the hydrophobic endgroup is coupled to one of the monomer units by a —S(CH₂)ₙO— group, where n is between 2 and about 24.

8. The copolymer of claim 1 which is a copolymer of N-isopropylacrylamide with acrylic acid, said copolymer having a C12 to C18 alkyl endgroup.

9. A micellar solution comprising micelles of an amphiphilic copolymer according to claim 1 in a liquid.

10. The micellar solution of claim 9 wherein the liquid is an organic liquid.

11. The micellar solution of claim 9 wherein the micelles comprise a core-shell structure in which a hydrophilic core is surrounded by a hydrophobic shell.

12. The micellar solution of claim 11 wherein a biological substance is located in the core of the micelles.

13. The micellar solution of claim 12 wherein the biological substance is an enzyme.

14. The micellar solution of claim 12 wherein the biological substance is catalytically active.

15. A process for making an amphiphilic copolymer comprising the step of:
coupling a precursor copolymer to an endcapping reagent so as to attach a hydrophobic endgroup to the precursor copolymer to form the amphiphilic copolymer,
wherein the precursor copolymer is a copolymer of a first monomer and a second monomer and the endcapping reagent comprises the hydrophobic endgroup, said first monomer being such that the amphiphilic copolymer is thermally responsive and said second monomer comprising a carboxylic acid or carboxylate group.

16. The process of claim 15 additionally comprising the step of:
copolymerising the first monomer and the second monomer by a free radical polymerisation to form the precursor copolymer.

17. The process of claim 16 wherein the step of copolymerising is conducted in the presence of a chain transfer agent, said chain transfer agent comprising a functional group capable of coupling to the endcapping reagent.

18. The process of claim 15 wherein the precursor copolymer has a hydroxyl endgroup and the endcapping reagent comprises a halogen.

19. A process for making a micellar solution comprising the step of combining an amphiphilic copolymer according to claim 1 and a liquid so as to form micelles of the copolymer in the liquid.

20. The process of claim 19 wherein the liquid is an organic liquid, whereby the micelles adopt a core-shell structure in which a hydrophilic core is surrounded by a hydrophobic shell.

21. A process for making a micellar solution comprising the steps of:
combining an amphiphilic copolymer according to claim 1 and an organic liquid so as to form micelles of the copolymer in the liquid, whereby the micelles adopt a core-shell structure in which a hydrophilic core is surrounded by a hydrophobic shell; and
adding a biological substance to the organic liquid so as to form the micellar solution wherein the biological substance is located in the core of the micelles.

22. The process of claim 21 wherein the biological substance is added as a solution, suspension, emulsion or microemulsion in a second liquid.

23. The process of claim 22 wherein the second liquid is an aqueous liquid.

24. A method for separating a biological substance from a micellar solution, wherein the micellar solution comprises micelles of an amphiphilic copolymer according to claim 1 in an organic liquid, said micelles comprising a core-shell structure in which a hydrophilic core is surrounded by a hydrophobic shell and the biological substance is located in the core of the micelles, said method comprising the step of heating the micellar solution to a temperature above the lower critical solution temperature of the copolymer.

25. A method for conducting a reaction of at least one reagent to produce a product, said method comprising the step of combining said at least one reagent with a micellar solution, said micellar solution comprising micelles of an amphiphilic copolymer according to claim 1 in an organic liquid, whereby the micelles comprise a core-shell structure in which a hydrophilic core is surrounded by a hydrophobic shell and a biological substance is located in the core of the micelles, said biological substance being capable of catalysing the reaction.

26. The method of claim 25 wherein the biological substance is an enzyme, whereby the method is a method for conducting an enzymatic reaction.

27. The method of claim 25 additionally comprising the step of separating the biological substance from the micellar solution by heating the micellar solution to a temperature above the lower critical solution temperature of the copolymer, said step being conducted after at least some of the at least one reagent has been reacted to produce the product.
